(19) United States
(12) Patent Application Publication
(10) Pub. No.: US 2008/0214672 A1
(43) Pub. Date: Sep. 4, 2008

(54) Treatment of Asthma with Aryl Sulfonamides

(76) Inventor: John Jessen Wright, Sandringham (AU)

Correspondence Address: BRINKS HOFER GILSON & LIONE, P.O. BOX 10395, CHICAGO, IL, 60610, US

(21) Appl. No.: 11/958,647

(22) Filed: Dec. 18, 2007

Related U.S. Application Data
(60) Provisional application No. 60/875,516, filed on Dec. 18, 2006.

(51) Int. Cl. A61K 31/145; A61K 31/136; A61P 11/06

(52) U.S. Cl. 514/604; 514/601

(57) ABSTRACT

The present invention relates to compounds that modulate various chemokine receptors. These compounds are useful for treating asthma.

### RELATED APPLICATIONS

This application claims priority to U.S. provisional application Ser. No. 60/875,516 filed Dec. 18, 2006. The disclosure of the priority application is incorporated herein in its entirety.

### BACKGROUND OF THE INVENTION

The present invention provides compounds, pharmaceutical compositions containing one or more of those compounds or their pharmaceutically acceptable salts, which are effective in inhibiting the binding or function of various chemokines, such as TECK (thymus-expressed chemokine), to the CCR9 receptor. As antagonists or modulators for the CCR9 receptor, the compounds and compositions have utility in treating asthma.

Asthma is a very common lung disease with the following characteristics: airways obstruction—this is usually reversible but often progressive chronic bronchial inflammation—a condition characterised by inflammatory cell infiltration and activation, release of biochemical mediators and structural changes (airway remodelling) bronchial hyperresponsiveness (BHR)—an exaggerated bronchoconstrictor response to a variety of immunologic, biochemical and physical stimuli.

Asthma is characterised clinically by chronic, intermittent airway obstruction with wheezing, coughing and breathlessness. Asthma is routinely diagnosed (See Guidelines for the diagnosis and development of asthma, 1997, NIH Publication No. 97-4051).

Chemokines and chemoline receptors have long thought to play a role in asthma (Carpenter et al., British J. Pharma. 2005, 145:1160-2). Recent work has shown that invariant NKT cells are potentially important in the pathogenesis of asthma (Jinquan et al., Arch. Immunol. Ther. Exp. 2006, 54:1-6; Akbari et al., N. Engl. J. Med. 2006, 354(11):1117-29 and Akbari et al., Nature Med. 2003, 9(5):582-88). In addition, Sen et al. (J. Immunol. 2005, 175:4914-26) showed that these invariant NKT cells express CCR9 and can bias normal T cells to produce Th2 cytokines which are important for the pathogenesis of asthma. Kay et al, (N. Engl. N. Med. 2006, 354(11):1186-88) proposes two ways in which CCR9 positive T cells can influence the generation of asthma one way being in the circulation the other being in the lung itself.

### BRIEF SUMMARY OF THE INVENTION

The present invention is directed to compounds and pharmaceutically acceptable salts thereof, compositions, and methods useful in modulating CCR9 chemokine activity. The compounds and salts thereof, compositions, and methods described herein are useful in treating or preventing CCR9-mediated conditions or diseases, such as asthma.

Salts and N-oxides of these compounds are also within the scope of the invention.

In one embodiment, the inventive compounds are of the formula (I):

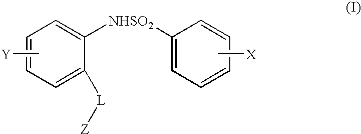

where X, Y, Z and L are as defined in U.S. publication no. 2005/137193, published Jun. 23, 2005, incorporated herein in its entirety.

In another embodiment, the inventive compounds are of the formula (II):

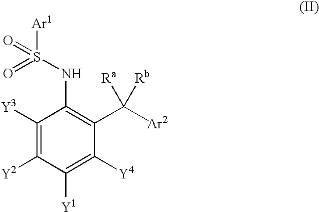

where Ar¹, Y¹, Y², Y³, Y⁴, R^{a}, R^{b} and Ar² are as defined in WO 2005/113513, published on Dec. 1, 2005, incorporated herein in its entirety.

In another embodiment, the inventive compounds are of the formula (III):

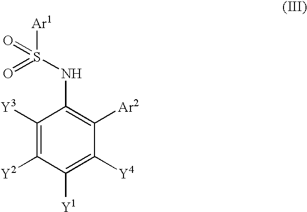

where Ar¹, Ar², Y¹, Y², Y³, and Y⁴ are as defined in WO 2005/113513, published on Dec. 1, 2005, incorporated herein in its entirety. Further compounds in accordance with this aspect of the invention are set forth in U.S. provisional application 60/831,042, filed Jul. 14, 2006, incorporated herein in its entirety.

In another embodiment, the inventive compounds are of the formula (IV):

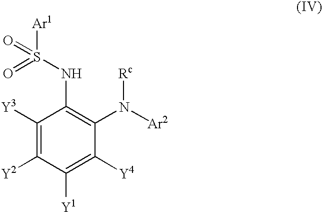

where Ar¹, Ar², R^{c}, Y¹, Y², Y³, and Y⁴ are as defined in WO 2005/113513, published on Dec. 1, 2005, incorporated herein in its entirety.

In another embodiment, the inventive compounds are of the formula (V):

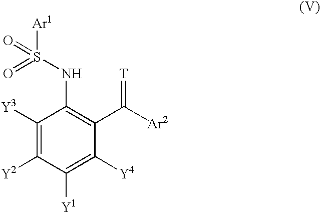

where Ar¹, Ar², T, Y¹, Y², Y³, and Y⁴ are as defined in WO 2005/113513, published on Dec. 1, 2005, incorporated herein in its entirety.

### DETAILED DESCRIPTION OF THE INVENTION

### Abbreviations and Definitions

When describing the compounds, compositions, methods and processes of this invention, the following terms have the following meanings, unless otherwise indicated.

“Pharmaceutically acceptable” means that the composition, carrier, diluent or excipient must be compatible with the other ingredients of the formulation and not deleterious to the recipient thereof.

“Therapeutically effective amount” refers to an amount sufficient to effect treatment when administered to a patient in need of treatment.

“Treating” or “treatment” as used herein refers to the treating or treatment of a disease or medical condition (such as a bacterial infection) in a patient, such as a mammal (particularly a human or a companion animal) which includes ameliorating the disease or medical condition, i.e., eliminating or causing regression of the disease or medical condition in a patient; suppressing the disease or medical condition, i.e., slowing or arresting the development of the disease or medical condition in a patient; or alleviating the symptoms of the disease or medical condition in a patient.

### Methods of Treating Asthma

The present invention provides methods of treating or preventing a asthma by administering to a subject having such a condition or disease a therapeutically effective amount of any compound of formulae (I, II, III, IV and V) or of compositions of these compounds. The “subject” is defined herein to include animals such as mammals, including, but not limited to, primates (e.g., humans), cows, sheep, goats, horses, dogs, cats, rabbits, rats, mice and the like. In preferred embodiments, the subject is a human.

The present invention is directed to the use of compounds, salts thereof, and compositions thereof to treate asthma, particularly the CCR9 component of this condition. Modulation of the CCR9 receptor activity, as used herein in its various forms, is intended to encompass antagonism, agonism, partial antagonism, inverse agonism and/or partial agonism of the activity associated with the CCR9 receptor. Accordingly, the compounds of the present invention are compounds which modulate at least one function or characteristic of mammalian CCR9, for example, a human CCR9 protein. The ability of a compound to modulate the function of CCR9, can be demonstrated in a binding assay (e.g., ligand binding or agonist binding), a migration assay, a signaling assay (e.g., activation of a mammalian G protein, induction of rapid and transient increase in the concentration of cytosolic free calcium), and/or cellular response assay (e.g., stimulation of chemotaxis, exocytosis or inflammatory mediator release by leukocytes).

Without intending to be bound by any particular theory, it is believed that the compounds provided herein interfere with the interaction between CCR9 and a CCR9 ligand, such as TECK.

The present invention provides compounds of the formula (I), (II), (III), (IV) and (V) as described above. Salts of these compounds are also contemplated.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, both solvated forms and unsolvated forms are intended to be encompassed within the scope of the present invention. Certain compounds of the present invention may exist in multiple crystalline or amorphous forms (i.e., as polymorphs). In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are intended to be encompassed within the scope of the present invention.

The present invention includes the use of compositions of these compounds for treating asthma. Generally, the compositions for modulating chemokine receptor activity in humans and animals will comprise a pharmaceutically acceptable excipient or diluent and a compound having the formula provided above as any of formulae (I, II, III, IV, and V).

The pharmaceutical compositions for the administration of the compounds of this invention may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing a compound of any of formulae (I, II, III, IV or V) can be formulated as described in WO 2005/113513, published on Dec. 1, 2005, incorporated herein in its entirety.

Depending on the subject's condition, the compounds and compositions of the present invention may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. The present invention also contemplates administration of the compounds and compositions of the present invention in a depot formulation.

In the treatment or prevention of conditions which require chemokine receptor modulation, an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5, 0.5 to 5.0, or 5.0 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In further embodiments, the present methods are directed to the treatment of asthma using a compound or composition of the invention in combination with a second therapeutic agent (preferably an anti-asthmatic compound, more specifically a P2-agonist or a corticosteroid).

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

### EXAMPLES

### Materials and Methods

### In Vitro Assays

### Reagents and Cells

MOLT-4 cells were obtained from the American Type Culture Collection (Manassas, Va.) and cultured in RPMI tissue culture medium supplemented with 10% fetal calf serum (FCS) in a humidified 5% CO₂ incubator at 37° C. Recombinant human chemokine protein TECK was obtained from R&D Systems (Minneapolis, Minn.). ChemoTX® chemotaxis microchambers were purchased from Neuro Probe (Gaithersburg, Md.). CyQUANT® cell proliferation kits were purchased from Molecular Probes (Eugene, Oreg.). Calcium indicator dye Fluo-4 AM was purchased from Molecular Devices (Mountain View, Calif.).

Conventional Migration Assay

Conventional migration assay was used to determine the efficacy of potential receptor antagonists in blocking migration mediated through CCR9. This assay was routinely performed using the ChemoTX® microchamber system with a 5-μm pore-sized polycarbonate membrane. To begin such an assay, MOLT-4 cells were harvested by centrifugation of cell suspension at 1000 PRM on a GS-6R Beckman centrifuge. The cell pellet was resuspended in chemotaxis buffer (H BSS with 0.1% BSA) at 5×10⁶ cells/mL. Test compounds at desired concentrations were prepared from 10 mM stock solutions by serial dilutions in chemotaxis buffer. An equal volume of cells and compounds were mixed and incubated at room temperature for 15 minutes. Afterwards, 20 μL of the mixture was transferred onto the porous membrane of a migration microchamber, with 29 μL of 50 nM chemokine TECK protein placed at the lower chamber. Following a 150-minute incubation at 37° C., during which cells migrated against the chemokine gradient, the assay was terminated by removing the cell drops from atop the filter. To quantify cells migrated across the membrane, 5 μL of 7× CyQUANT® solution was added to each well in the lower chamber, and the fluorescence signal measured on a Spectrafluor Plus fluorescence plate reader (TECAN, Durham, N.C.). The degree of inhibition was determined by comparing migration signals between compound-treated and untreated cells. IC50 calculation was further performed by non-linear squares regression analysis using Graphpad Prism (Graphpad Software, San Diego, Calif.).

RAM Assay

The primary screen to identify CCR9 antagonists was carried out using RAM assay (WO 02101350), which detects potential hits by their ability to activate cell migration under inhibitory TECK concentration. To begin such an assay, MOLT-4 cells were harvested by centrifugation of cell suspension at 1000 RPM on a GS-6R Beckman centrifuge. The cell pellet was resuspended in chemotaxis buffer (HBSS/0.1% BSA) at 5×10⁶ cells/mL. Twenty-five microliters of cells was mixed with an equal volume of a test compound diluted to 20 μM in the same buffer. Twenty microliters of the mixture was transferred onto the filter in the upper chemotaxis chamber, with 29 μL of 500 nM chemokine protein TECK placed in the lower chamber. Following a 150-minute incubation at 37° C., the assay was terminated by removing the cell drops from atop the filter. To quantify cells migrated across the membrane, 5 μL of 7× CyQUANT® solution was added to each well in the lower chamber, and the fluorescence signal measured on a Spectrafluor Plus fluorescence plate reader (TECAN, Durham, N.C.).

For selection of potential hits, the level of migration activation was calculated as a RAM index—the ratio between the signal of a particular well and the median signal of the whole plate. Compounds with a RAM index of greater than 1.8 were regarded as RAM positive, and were selected for IC₅₀ determinations in conventional functional assays.

Calcium Flux Assay

Calcium flux assay measures an increase in intracellular calcium following ligand-induced receptor activation. In the screen of CCR9 antagonists, it was used as a secondary assay carried out on a FLIPR® machine (Molecular Devices, Mountain View, Calif.). To begin an assay, MOLT-4 cells were harvested by centrifugation of cell suspension, and resuspended to 1.5×10⁶ cells/mL in HBSS (with 1% fetal calf serum). Cells were then labeled with a calcium indicator dye Fluo-4 AM for 45 minutes at 37° C. with gentle shaking. Following incubation, cells were pelletted, washed once with HBSS and resuspended in the same buffer at a density of 1.6×10⁶ cells/mL. One hundred microliters of labeled cells were mixed with 10 μL of test compound at the appropriate concentrations on an assay plate. Chemokine protein TECK was added at a final concentration of 25 nM to activate the receptor. The degree of inhibition was determined by comparing calcium signals between compound-treated and untreated cells. IC₅₀ calculations were further performed by non-linear squares regression analysis using Graphpad Prism (Graphpad Software, San Diego, Calif.).

### Hypothetical Examples

### Asthma Assay

Compounds of the present invention will be tested for anti-asthma activity as previously described (Carpenter et al., British J. Pharma. 2005, 145:1160-2). Briefly, in vitro and in vivo assays are described below.

### In Vitro Experiments

Specific pathogen free (SPF), female BALB/c mice will be purchased from Jackson Laboratories (Bar Harbor, Me., U.S.A.) and housed in the animal care facility. SPF mice will be euthanized and subjected toperitoneal lavages with 10 ml sterile saline. Lavages will be pooled, red blood cells will be lysed in ammonium chloride buffer, and the remaining cells will be thoroughly washed with saline. Cells will be counted and subjected to Diff-Quik staining to determine the number of peritoneal macrophages.

Cells will be resuspended in complete DMEM (BioWhittaker, Walkersville, Md., U.S.A.) containing 5% FCS, 2 mM L-glutamine, 100 U/ml penicillin, and 100 U/ml streptomycin.

Cells will be plated in plastic plates and incubated 1-2 h at 37 C in 5% CO2. Nonadherent cells will be removed and adherent cells will be washed with complete DMEM. The adherent macrophages will then be rested overnight in a CO2 incubator. Subsequently, vehicle (2.5% DMSO) or BX-471 at 10 mM in fresh DMEM will be added to separate tissue culture wells (in triplicate) containing approximately 5×10⁵ cells/well of a 24-well tissue culture plate for 24 h.

Compounds in accordance with the present invention will be given at a dose of 10 mM for the in vitro studies. At the conclusion of experiment, cell-free supernatants will be collected for ELISA analysis and the macrophages will be prepared for prepared for RNA isolation.

### In Vivo Testing

Murine model of chronic fungal-induced allergic airway disease or fungal asthma SPF, female CBA/J mice will be purchased from the Jackson Laboratories (Bar Harbor, Me., U.S.A.) and will be maintained in an SPF facility for the duration of this study. Systemic sensitization of mice to a commercially available preparation of soluble A. fumigatus antigens will be performed as previously described in detail (Hogaboam et al., Am. J. Pathol. 2000, 156:723-732). At 7 days after the third intranasal challenge, each mouse will receive 5.0×106 A. fumigatus conidia suspended in 30 ml of 0.1% Tween-80 via the intratracheal route (Hogaboam et al., 2000).

At day 14 after the A. fumigatus conidia challenge, compounds in accordance with the present invention will be given at a variety of dosages.

Measurement of Bronchial Hyper-Responsiveness

At day 28 after the A. fumigatus conidia challenge, bronchial hyper-responsiveness in treated mice will be measured in a Buxcot plethysmograph (Buxco, Troy, N.Y., U.S.A.) as previously described (Hogaboam et al., 2000). Sodium pentobarbital will be used to anesthetize each mouse prior to its intubation for ventilation with a Harvard pump ventilator (Harvard Apparatus, Reno, Nev., U.S.A.). The following ventilation parameters will be used: tidal volume=0.25 ml, breathing frequency=120 min⁻¹, and positive end-expiratory pressure ca. 3 cm H₂O. Within the sealed plethysmograph mouse chamber, transrespiratory pressure (i.e. Δ tracheal pressure-Δ mouse chamber pressure) and inspiratory volume or flow will be continuously monitored online by an adjacent computer, and airway resistance will be calculated by the division of the transpulmonary pressure by the change in inspiratory volume. At the conclusion of the assessment of airway responsiveness, a bronchoalveolar lavage (BAL) will be performed with 1 ml of normal saline. Approximately 500 ml of blood will then be removed from each mouse and transferred to a microcentrifuge tube. Sera will obtained after the sample is centrifuged at 10,000 r.p.m. for 5 min. Whole lungs will finally be dissected from each mouse and snap frozen in liquid N₂ or prepared for histological analysis.

Morphometric Analysis of Leukocyte Accumulation in BAL Samples

Lymphocytes and macrophages will be enumerated in BAL samples cytospun (Shandon Scientific, Runcorn, U.K.) onto coded microscope slides. Each slide will be stained with a Wright-Giemsa differential stain, and the average number of each cell type will be determined.

Whole Lung Histological Analysis

Whole lung samples from all treatment groups at day 30 after A. fumigatus conidia challenge will be fully inflated with 10% formalin, dissected and placed in fresh formalin for 24 h. Routine histological techniques will be used to paraffin-embed the entire lung, and 5 mm sections of whole lung were stained with Gomori methenamine silver (GMS), hematoxylin and eosin (H&E), or with Periodic Acid Schiff (PAS). Inflammatory infiltrates and structural alterations will be examined around small airways and adjacent blood vessels using light microscopy.

SuperArray Analysis and Quantitative TAQMAN Polymerase Chain Reaction (PCR) Analysis

SuperArray analysis of transcript expression will be performed according to the directions provided with these kits (GEArrayt Q series KIT nonradioactive; SuperArray Inc., Bethesda, Md., U.S.A.). Briefly, from cultured macrophages or homogenized mouse lungs, total RNA will be isolated using the Trizol reagent (Invitrogen/Life Technologies, Carlsbad, Calif., U.S.A.). Once isolated, 1-2.5 mg of total RNA, pooled from the in vitro and in vivo treatment groups, will be used as the template for Biotin-labeled cDNA probe synthesis. The labeled probes will then be hybridized to the mouse inflammatory cytokine/receptor GEArrayt Q series membrane containing 96 genes related to murine cytokine and chemokine ligands and receptors. After an overnight incubation at 60° C., the membranes will be washed several times, blocked, and subjected to chemiluminescent detection (alkaline phosphatase-conjugated streptavidin; 1:10,000 dilution) with the chemiluminescent substrate for alkaline phosphatase, phenylphosphate substituted 1,2 dioxetane (CDP-star). After exposing the membranes to X-ray film, the developed films will be scanned to create raw image files, which will be analyzed using an image analysis software program (Scanalyze by Michael Eisen). The relative abundance of a particular transcript will be estimated by directly comparing its signal intensity to the signal derived from three or four combined housekeeping genes.

ELISA Analysis

Murine CCL3, CCL5, CCL6, CCL22, TNF-a, IL-4, IL-5, IL-12, IL-13, IFN-g, and IL-10 levels will be measured in 50-ml samples from cell-free supernatants (from macrophage cultures) and/or whole lung homogenates using a standardized sandwich ELISA technique previously described in detail (Evanoff et al., 1992). Each ELISA will be screened to ensure antibody specificity and recombinant murine cytokines, and chemokines were used to generate the standard curves from which the concentrations present in the samples were derived.

1. A method comprising administering a therapeutically effective amount of compound of the formula (I), (II), (III), (IV) or (V) to an asthma patient in need thereof.
